(19) United States
(12) Patent Application Publication
(10) Pub. No.: US 2023/0025577 A1
(43) Pub. Date: Jan. 26, 2023

(54) SYSTEM, METHOD AND SENSOR DEVICE FOR SENSING A CHANGE IN A CONCENTRATION OF MICRO-ORGANISMS

(71) Applicant: FRAUNHOFER UK RESEARCH LTD, Glasgow (GB)

(72) Inventors: Mark SCULLION, Glasgow (GB); Anne-Marie HAUGHEY, Glasgow (GB)

(73) Assignee: FRAUNHOFER UK RESEARCH LTD, Glasgow (GB)

(21) Appl. No.: 17/784,512

(22) PCT Filed: Dec. 11, 2020
(86) PCT No.: PCT/EP2020/085838

§ 371 Date: Jun. 10, 2022

(30) Foreign Application Priority Data
Dec. 12, 2019 (GB) 1918326.8

(51) Int. Cl. B01L 3/00; G01N 21/45; C12Q 1/18; C12Q 1/06

(52) U.S. Cl. CPC B01L 3/502761; G01N 21/45; B01L 3/502715; C12Q 1/18; C12Q 1/06; G01N 2021/458; B01L 2200/0652; B01L 2300/0663; B01L 2300/046

(57) ABSTRACT

A sensor device for use in sensing a change in a concentration of micro-organisms, comprises a waveguide interferometer having a sensing arm and a reference arm, a microfluidic channel for a fluid containing the micro-organisms, and a trapping arrangement in the microfluidic channel for physically trapping the micro-organisms when the fluid flows along the microfluidic channel so as to concentrate the micro-organisms in a sensing region of the microfluidic channel. The sensing arm is configured to guide sensing light, the reference arm is configured to guide reference light, and the waveguide interferometer is configured to interfere the sensing light with the reference light. The waveguide interferometer and the microfluidic channel are configured to allow the sensing light to interact with the fluid and the micro-organisms in the sensing region of the microfluidic channel.

### CROSS-REFERENCE TO RELATED APPLICATIONS

This application is a National Stage Patent Application (filed under 35 § U.S.C. 371) of PCT/EP2020/085838, filed Dec. 11, 2020, of the same title, which, in turn claims priority to Great Britain Patent Application No. 1918326.8 filed Dec. 12, 2019, of the same title; the contents of each of which are hereby incorporated by reference.

### FIELD OF THE INVENTION

The present disclosure relates to a system, method and sensor device for sensing a change in a concentration of micro-organisms such as bacteria, for example as a result of growth of the micro-organisms and, in particular though not exclusively, for sensing the susceptibility of the growth of the micro-organisms to one or more micro-organism growth-inhibiting substances such as one or more antibiotics. The present disclosure also relates to a reader apparatus for reading the sensor device.

### BACKGROUND OF THE INVENTION

It is known for clinicians such as doctors to send-off samples of bodily fluids such as urine, blood or the like to a central hospital lab, where culture-based tests can determine bacterial counts, identify bacterial species and their antibiotic susceptibility. However, these tests can often take several days and, therefore, it is not uncommon for an antibiotic to be prescribed before testing, which is later found out to be the wrong one or unnecessary. This may lead to the faster evolution of bacteria to develop resistance to antibiotics, which has dire implications for the future of healthcare worldwide. A recent Review on Antimicrobial Resistance recommended that all prescriptions be backed up by diagnostic testing by 2020.

### SUMMARY OF THE INVENTION

It should be understood that any one or more of the features of any one of the following aspects of the present disclosure may be combined with any one or more of the features of any of the other following aspects of the present disclosure.

According to at least one aspect of the present disclosure there is provided a sensor device for use in sensing a change in a concentration of micro-organisms, the sensor device comprising:

a waveguide interferometer having a sensing arm and a reference arm;

a microfluidic channel for a fluid containing micro-organisms; and

a trapping arrangement in the microfluidic channel for physically trapping micro-organisms when the fluid flows along the microfluidic channel so as to concentrate the micro-organisms in a sensing region of the microfluidic channel,

wherein the sensing arm is configured to guide sensing light, the reference arm is configured to guide reference light, and the waveguide interferometer is configured to interfere the sensing light with the reference light, and

wherein the waveguide interferometer and the microfluidic channel are configured to allow the sensing light to interact with the fluid and the micro-organisms in the sensing region of the microfluidic channel.

Any change in the optical path length difference between the sensing arm and the reference arm as a concentration of the micro-organisms in the fluid in the sensing region of the microfluidic channel changes, results in a change in the phase difference between the sensing light and the reference light and therefore a change in the intensity of the light at an output of the waveguide interferometer. Consequently, the evolution of the intensity of the light at an output of the waveguide interferometer as a function of time may provide a measure of the change in concentration of the micro-organisms in the fluid in the sensing region of the microfluidic channel. Consequently, the sensor device may be used for measuring a change in a concentration of micro-organisms in a fluid. In particular, the sensor device may be used for measuring a growth or a decline of the micro-organisms in the fluid. The sensor device may be used for measuring a rate of change in the concentration of micro-organisms in the fluid.

The micro-organisms may comprise at least one of bacteria, fungi and algae.

The fluid may be a bodily fluid such as urine, blood, saliva, sputum or the like.

The fluid may be a non-bodily fluid.

The fluid may be water.

The sensing arm may be configured to guide the sensing light as a guided optical mode.

The sensing arm and the microfluidic channel may be configured to allow an evanescent field of the guided optical mode to interact with the bacteria in the sensing region.

The sensing arm may comprise an optical waveguide such as a single-mode optical waveguide.

The reference arm may be configured to guide the reference light as a guided optical mode.

The reference arm may comprise an optical waveguide such as a single-mode optical waveguide.

The guided optical mode in the optical waveguide of each of the sensing and reference arms may comprise a guided transverse magnetic (TM) optical mode. Use of a TM optical mode may provide greater measurement sensitivity than a transverse electric (TE) optical mode because a TM optical mode is less well confined and thus interacts more with the micro-organisms in the sensing region of the microfluidic channel. Also, use of a TM optical mode is more tolerant to fabrication imperfections in width and sidewall roughness of waveguides in the sensing and reference arms.

The guided optical mode in the optical waveguide of each of the sensing and reference arms may comprise a guided transverse electric (TE) optical mode.

The trapping arrangement may be defined by the sensing arm.

The trapping arrangement may comprise one or more breaks or gaps in the sensing arm. The one or more breaks or gaps in the sensing arm may be configured to trap and/or accommodate micro-organisms whilst permitting fluid to flow through the one or more breaks or gaps in the sensing arm.

The one or more breaks or gaps in the sensing arm may be defined in a waveguide core of the sensing arm. The waveguide interferometer and the microfluidic channel may be configured so that the one or more breaks or gaps in the sensing arm are located in the sensing region of the microfluidic channel to allow the sensing light to propagate through any micro-organisms located in any of the breaks or gaps in the sensing arm.

The waveguide core of the sensing arm may be defined by a plurality of holes or pillars. Such a waveguide core may act as a photonic crystal waveguide. The trapping arrangement may be defined by the plurality of holes or pillars.

The one or more breaks or gaps in the sensing arm may be defined in a waveguide cladding of the sensing arm. The one or more breaks or gaps in the sensing arm may be defined in an upper waveguide cladding of the sensing arm and/or in a lower waveguide cladding of the sensing arm.

The trapping arrangement may be defined by a path of the sensing arm. For example, the sensing arm waveguide may follow a path that defines one or more regions or bays for trapping and/or accommodating micro-organisms to one side of the sensing arm waveguide. For example, the sensing arm waveguide may follow a serpentine, sinusoidal or square wave path that defines one or more regions or bays for trapping and/or accommodating micro-organisms to one side of the sensing arm waveguide. The sensing arm may define one or more gaps or breaks in the sensing arm to permit fluid to flow through the one or more breaks or gaps in the sensing arm.

The waveguide interferometer and the microfluidic channel may be configured to allow the reference light to interact with the fluid and the micro-organisms in the microfluidic channel. The waveguide interferometer and the microfluidic channel may be configured for exposure of the reference arm of the waveguide interferometer to the fluid and the micro-organisms. In use, the concentration of micro-organisms in the vicinity of the reference arm may be much lower than the concentration of micro-organisms in the sensing region in the vicinity of the sensing arm. Configuring the waveguide interferometer and the microfluidic channel to allow the reference light to interact with the fluid and the micro-organisms in the microfluidic channel or for exposure of the reference arm of the waveguide interferometer to the fluid and the micro-organisms, may result in a simpler sensor device and/or a sensor device which is more easily manufactured because there is no need to include an additional cover layer or mask to prevent exposure of the reference arm to the fluid and the micro-organisms in the microfluidic channel.

The waveguide interferometer and the microfluidic channel may be configured so as to prevent the reference light from interacting with the fluid and the micro-organisms in the microfluidic channel. The waveguide interferometer and the microfluidic channel may be configured so as to prevent exposure of the reference arm to the fluid containing the micro-organisms. The sensor device may comprise a cover layer or mask located between the reference arm and the microfluidic channel, which cover layer or mask prevents the reference light from interacting with the fluid and the micro-organisms in the microfluidic channel. The cover layer or mask may prevent exposure of the reference arm to the fluid containing the micro-organisms. By preventing the reference light from interacting with the fluid and the micro-organisms in the microfluidic channel or preventing exposure of the reference arm to the fluid containing the micro-organisms, the effective refractive index difference between the sensing and reference arms and, therefore, the phase difference between the sensing and reference light may be greater so that the sensor device may provide a more sensitive measurement, or a more accurate measurement, of the change in the concentration of micro-organisms.

The sensing and reference arms may be symmetric i.e. the sensing and reference arms may have the same length. The sensing and reference arms may be balanced i.e. the sensing and reference arms may have the same optical path length. For example, the sensing and reference arms may be formed from the same materials, may have the same cross-sectional geometries and may be of the same length. The use of balanced sensing and reference arms may be better in terms of thermal stability i.e. the use of balanced sensing and reference arms may reduce any change in the intensity of the light at the output of the waveguide interferometer as a result of a change in temperature. The use of balanced sensing and reference arms may also help to cancel out any refractive index changes that are not due to a change in concentration of micro-organisms in the sensing region. For example, when the waveguide interferometer and the microfluidic channel are configured to allow the reference light to interact with the fluid and the micro-organisms in the microfluidic channel, the use of balanced sensing and reference arms may also help to cancel out any refractive index change of the fluid that is not caused by a change in concentration of micro-organisms in the sensing region.

The sensing and reference arms may be asymmetric i.e. the sensing and reference arms may have different lengths. The sensing and reference arms may be unbalanced i.e. the sensing and reference arms may have different optical path lengths. The use of unbalanced sensing and reference arms may be more sensitive to the change in concentration of micro-organisms in the sensing region, but is potentially less stable to changes in temperature. The use of unbalanced sensing and reference arms may allow the intensity of the light at the output of the waveguide interferometer to be measured as a function of wavelength either using a spectrally broadband optical source and an optical spectrometer or using a tuneable optical source such as a tuneable laser and a photodetector. Changes of the concentration of the micro-organisms as a function of time may be determined from changes over time in the intensity of the light at the output of the waveguide interferometer as a function of wavelength. For example, a change in concentration of the micro-organisms may result in a change in wavelength periodicity or free spectral range of the intensity of the light at the output of the waveguide interferometer as a function of wavelength. Consequently, the use of unbalanced sensing and reference arms may allow the free spectral range of the intensity of the light at the output of the waveguide interferometer to be measured repeatedly at different times and allow a change of concentration of the micro-organisms over time to be determined from the repeated measurements of the free spectral range.

The sensing arm may be folded so that the sensing arm passes the sensing region of the microfluidic channel a plurality of times. This may increase the overall change in phase experienced by the sensing light in the sensing arm and thereby increase the sensitivity of the measurement of the change in concentration of the micro-organisms.

The reference arm of each waveguide interferometer may be folded.

The sensor device may comprise:

a plurality of waveguide interferometers, each waveguide interferometer having a sensing arm and a reference arm;

a plurality of microfluidic channels for the fluid and the micro-organisms, and

a trapping arrangement in each microfluidic channel for physically trapping the micro-organisms when the fluid flows along the corresponding microfluidic channel so as to concentrate the micro-organisms in a corresponding sensing region.

Each sensing arm may be configured to guide sensing light, each reference arm may be configured to guide reference light, and each waveguide interferometer may be configured to interfere the corresponding sensing light with the corresponding reference light. The waveguide interferometers and the microfluidic channels may be configured to allow the sensing light in the sensing arm of each waveguide interferometer to interact with the fluid and the micro-organisms in the sensing region of the corresponding microfluidic channel.

One of the microfluidic channels may contain a first micro-organism growth-inhibiting substance. Such a sensor device may allow a measurement of the susceptibility of the micro-organisms to the first micro-organism growth-inhibiting substance.

Said one of the microfluidic channels may contain the first micro-organism growth-inhibiting substance at a position located upstream from the corresponding sensing region in said one of the microfluidic channels.

One or more of the other microfluidic channels may contain a corresponding micro-organism growth-inhibiting substance which is different to the first micro-organism growth-inhibiting substance. Such a sensor device may allow the susceptibility of the micro-organisms to different micro-organism growth-inhibiting substances.

One or more of the other microfluidic channels may contain a corresponding micro-organism growth-inhibiting substance at a position located upstream from the corresponding sensing region, which corresponding micro-organism growth-inhibiting substance is different to the first micro-organism growth-inhibiting substance.

One or more of the other microfluidic channels may not contain any micro-organism growth-inhibiting substance. Any one of the microfluidic channels which does not contain any micro-organism growth-inhibiting substance may serve as a reference microfluidic channel. Specifically, a measurement of the intensity of the light as a function of time at the output of a waveguide interferometer corresponding to a microfluidic channel which contains a micro-organism growth-inhibiting substance may be compared to a measurement of the intensity of the light as a function of time at the output of a waveguide interferometer corresponding to the reference microfluidic channel so as to provide a relative measurement of the susceptibility of the growth of the micro-organisms in the microfluidic channel which contains the micro-organism growth-inhibiting substance. In the case of the reference microfluidic channel without any micro-organism growth-inhibiting substance, the intensity of the light at the output of the corresponding waveguide interferometer may trace out a series of interference fringes as the micro-organisms grow i.e. the intensity of the light as a function of time at the output of the waveguide interferometer corresponding to the reference microfluidic channel may be generally oscillatory. However, if a micro-organism growth-inhibiting substance is effective in another microfluidic channel such that the growth of micro-organisms in the other microfluidic channel slows, the interference fringes for the other microfluidic channel may be longer than the interference fringes for the reference microfluidic channel. If a micro-organism growth-inhibiting substance is effective in another microfluidic channel such that the growth of micro-organisms in the other microfluidic channel stops, the interference fringes in the other microfluidic channel may effectively disappear.

Each microfluidic channel may contain a different micro-organism growth-inhibiting substance.

Only one of the microfluidic channels may not contain any micro-organism growth-inhibiting substance.

The micro-organisms may comprise bacteria and each micro-organism growth-inhibiting substance may comprise an antibiotic.

Each microfluidic channel may comprise a well such as a through-hole or recess for receiving a micro-organism growth-inhibiting substance. The well may be located at a position upstream from the corresponding sensing region in the same microfluidic channel.

Each trapping arrangement may be located downstream from the sensing arm of the corresponding waveguide interferometer.

Each trapping arrangement may be located at the same position along the corresponding microfluidic channel as the sensing arm of the corresponding waveguide interferometer.

Each trapping arrangement may be located adjacent to the sensing arm of the corresponding waveguide interferometer. For example, each trapping arrangement may be located over, above, on top of, under, below, underneath and/or beside the sensing arm of the corresponding waveguide interferometer.

The trapping arrangement in each microfluidic channel may define one or more gaps which are configured to allow fluid flow to pass the trapping arrangement but to prevent micro-organisms from passing the trapping arrangement.

Each waveguide interferometer may be defined on, or adjacent, a surface of a photonic chip. The trapping arrangement may define one or more gaps between the trapping arrangement and the surface of the photonic chip, wherein each gap is configured to allow fluid flow to pass through the gap between the trapping arrangement and the surface of the photonic chip but to prevent micro-organisms from passing through the gap between the trapping arrangement and the surface of the photonic chip.

The trapping arrangement in each microfluidic channel may comprise a plurality of trapping features, wherein the trapping features are configured to physically trap the micro-organisms when the fluid flows along the microfluidic channel.

The trapping features may define one or more gaps which are configured to allow fluid flow to pass the trapping features but to prevent micro-organisms from passing the trapping features.

Each trapping feature may define one or more gaps between the trapping feature and the surface of the photonic chip, wherein each gap is configured to allow fluid flow to pass through the gap between the trapping feature and the surface of the photonic chip but to prevent micro-organisms from passing through the gap between the trapping feature and the surface of the photonic chip.

The trapping arrangement in each microfluidic channel may comprise a row of trapping features.

The trapping arrangement in each microfluidic channel may comprise two or more rows of trapping features.

The two or more rows of trapping features may be staggered.

Each trapping feature may comprise a trap configured to physically trap the micro-organisms when the fluid flows along the microfluidic channel.

Each trap may comprise one or more features extending into the corresponding microfluidic channel so as to define a bay in the corresponding microfluidic channel for accommodating one or more micro-organisms.

The sensing arm of each waveguide interferometer may be folded so that the sensing arm passes the corresponding sensing region of the corresponding microfluidic channel a plurality of times. This may increase the overall change in phase experienced by the sensing light in the sensing arm and thereby increase the sensitivity of the measurement of the change in concentration of the micro-organisms.

The reference arm of each waveguide interferometer may be folded.

The sensing and reference arms of each waveguide interferometer may be symmetric i.e. the sensing and reference arms may have the same length. The sensing and reference arms of each waveguide interferometer may be balanced i.e. the sensing and reference arms of each waveguide interferometer may have the same optical path length. For example, the sensing and reference arms of each waveguide interferometer may be formed from the same materials, may have the same cross-sectional geometries and may be of the same length. The use of balanced sensing and reference arms may be better in terms of thermal stability i.e. the use of balanced sensing and reference arms may reduce any change in the intensity of the light at the output of each waveguide interferometer as a result of a change in temperature. The use of balanced sensing and reference arms may also help to cancel out any refractive index changes that are not due to a change in concentration of micro-organisms in each sensing region. For example, when each waveguide interferometer and the corresponding microfluidic channel are configured to allow the reference light to interact with the fluid and the micro-organisms in the corresponding microfluidic channel, the use of balanced sensing and reference arms may also help to cancel out any refractive index change of the fluid that is not caused by a change in concentration of micro-organisms in the sensing region of the corresponding microfluidic channel.

The sensing and reference arms of each waveguide interferometer may be asymmetric i.e. the sensing and reference arms may have different lengths. The sensing and reference arms of each waveguide interferometer may be unbalanced i.e. the sensing and reference arms of each waveguide interferometer may have different optical path lengths. The use of unbalanced sensing and reference arms may be more sensitive to the change in concentration of micro-organisms in the sensing region of each microfluidic channel, but is potentially less stable to changes in temperature. The use of unbalanced sensing and reference arms may allow the intensity of the light at the output of each waveguide interferometer to be measured as a function of wavelength either using a spectrally broadband optical source and an optical spectrometer or using a tuneable optical source such as a tuneable laser and a photodetector. Changes of the concentration of the micro-organisms as a function of time may be determined from changes over time in the intensity of the light at the output of each waveguide interferometer as a function of wavelength. For example, a change in concentration of the micro-organisms may result in a change in wavelength periodicity or free spectral range of the intensity of the light at the output of each waveguide interferometer as a function of wavelength. Consequently, the use of unbalanced sensing and reference arms may allow the free spectral range of the intensity of the light at the output of each waveguide interferometer to be measured repeatedly at different times and allow a change of concentration of the micro-organisms over time to be determined from the repeated measurements of the free spectral range of each waveguide interferometer.

The sensor device may comprise a filtering arrangement in each microfluidic channel at a position located upstream from the corresponding sensing region, wherein the filtering arrangement is configured to trap debris or particulates which are greater in size than the micro-organisms, for example debris or particulates having a minimum dimension which is greater than a maximum dimension of the micro-organisms.

Each filtering arrangement may comprise one or more projections such as one or more cylindrical pillars extending into the corresponding microfluidic channel, wherein the one or more projections define at least one gap that exceeds a maximum dimension of the micro-organisms.

Each waveguide interferometer may be defined by a photonic chip.

Each microfluidic channel may be defined by a microfluidic chip.

The microfluidic chip may comprise a fluid inlet for the injection of fluid into one or more of the microfluidic channels.

The photonic chip and the microfluidic chip may be aligned so as to align the sensing arm of each waveguide interferometer with the sensing region of a corresponding microfluidic channel.

The microfluidic chip may be configured so that each filtering arrangement is located the same distance from the fluid inlet. This means that the fluid and the micro-organisms should reach the filtering arrangement in each microfluidic channel at the same time when the fluid and the micro-organisms are injected into a plurality of the microfluidic channels via the fluid inlet.

The microfluidic chip may be configured so that each well for receiving an antibiotic is located the same distance from the fluid inlet. This means that the fluid and the micro-organisms should reach the well in each microfluidic channel at the same time when the fluid and the micro-organisms are injected into a plurality of the microfluidic channels via the fluid inlet.

The microfluidic chip may be configured so that each trapping arrangement is located the same distance from the fluid inlet. This means that the fluid and the micro-organisms should reach the trapping arrangement in each microfluidic channel at the same time when the fluid and the micro-organisms are injected into a plurality of the microfluidic channels via the fluid inlet.

The photonic chip may define one or more optical outputs, each optical output connected to an output waveguide of a corresponding waveguide interferometer.

Each output waveguide may be a single-mode output waveguide.

The photonic chip may define a single optical input.

The photonic chip may define an input waveguide that extends from the single optical input. The input waveguide may be a single-mode input waveguide.

The photonic chip may define one or more waveguide couplers or waveguide splitters that connect the input waveguide of the photonic chip to an input waveguide of each of the waveguide interferometers. If the coupling of light from an optical source into the single optical input of the photonic chip varies or if an output optical power of the optical source varies, the use of a single optical input may ensure that the ratio of the optical power levels at the inputs of the different waveguide interferometers remains stable or constant.

The photonic chip may define a reference waveguide, wherein one of the optical outputs of the photonic chip is connected to the reference waveguide. The reference waveguide may be a single-mode reference waveguide.

The waveguide couplers or waveguide splitters may connect the single optical input of the photonic chip to the reference waveguide. Such a reference waveguide may be used to monitor fluctuations in the coupling of light from an optical source into the single optical input of the photonic chip or fluctuations in the output optical power of the optical source and to normalize the optical intensities at the outputs of the different waveguide interferometers accordingly.

The single optical input may be located at a first edge of the photonic chip and the one or more optical outputs may be located at a second edge of the photonic chip opposite to the first edge.

The single optical input and the one or more optical outputs may be located at the same edge of the photonic chip.

The photonic chip may define at least one bend in at least one of the input waveguide, the output waveguides and the reference waveguide.

The photonic chip may comprise or be formed from a material which is not absorbing at a wavelength of the light propagating through each waveguide interferometer.

The photonic chip may comprise, or be formed from, at least one of a silicon-on-insulator material, silica or glass, a polymer material, and silicon nitride.

The photonic chip may be disposable.

The microfluidic chip may comprise, or be formed from, at least one of polydimethylsiloxane (PDMS), silica or glass, a polymer material, silicon, and silicon nitride.

The microfluidic chip may be disposable.

According to at least one aspect of the present disclosure there is provided a sensor device for use in sensing a change in a concentration of micro-organisms, the sensor device comprising:

a plurality of waveguide interferometers, each waveguide interferometer having a sensing arm and a reference arm; and

a plurality of microfluidic channels, each channel configured to accommodate a fluid containing micro-organisms,

wherein each sensing arm is configured to guide sensing light, each reference arm is configured to guide reference light, and each waveguide interferometer is configured to interfere the corresponding sensing light with the corresponding reference light, and

wherein each waveguide interferometer and the corresponding microfluidic channel are configured so that the sensing light of each waveguide interferometer interacts with a greater concentration of the micro-organisms in the corresponding microfluidic channel than the corresponding reference light,

wherein one of the microfluidic channels contains a first micro-organism growth-inhibiting substance, and

wherein one or more of the other microfluidic channels contains a corresponding micro-organism growth-inhibiting substance which is different to the first micro-organism growth-inhibiting substance and/or one or more of the other microfluidic channels does not contain any micro-organism growth-inhibiting substance.

Each microfluidic channel may contain a different micro-organism growth-inhibiting substance.

Only one of the microfluidic channels may not contain any micro-organism growth-inhibiting substance.

One of the microfluidic channels may contain the first micro-organism growth-inhibiting substance at a position located upstream in the microfluidic channel from the corresponding sensing arm.

One or more of the other microfluidic channels may contain a corresponding micro-organism growth-inhibiting substance at a position located upstream from the corresponding sensing arm, which corresponding micro-organism growth-inhibiting substance is different to the first antibiotic.

The sensor device may comprise a trapping arrangement in each microfluidic channel for physically trapping the micro-organisms when the fluid flows along the microfluidic channel so as to concentrate the micro-organisms in a corresponding sensing region of the microfluidic channel.

Each waveguide interferometer and the corresponding microfluidic channel may be configured to allow the sensing light to interact with the fluid and the micro-organisms in the sensing region of the corresponding microfluidic channel.

According to at least one aspect of the present disclosure there is provided a reader apparatus for reading a sensor device as described above, the reader apparatus comprising:

an optical source for emitting light to be coupled into each waveguide interferometer;

one or more optical detectors for detecting light output from each waveguide interferometer and generating a corresponding electrical signal; and

a controller for determining a change, or a rate of change, in the concentration of the micro-organisms in the sensing region of each microfluidic channel based on the evolution of the corresponding electrical signal over time.

As the concentration of the micro-organisms in the sensing region of a sensing arm of a waveguide interferometer corresponding to a particular microfluidic channel changes, for example as a consequence of growth or decline in the micro-organisms in the sensing region, the optical path length difference and therefore also the phase difference between the sensing and reference light in the corresponding waveguide interferometer changes. Consequently, the intensity of the light at the output of each waveguide interferometer may oscillate and the corresponding electrical signal detected by the corresponding optical detector may oscillate as the concentration of the micro-organisms in the sensing region of the corresponding microfluidic channel changes, for example as a consequence of growth or decline in the micro-organisms in the sensing region.

The controller may be configured to determine a change, or a rate of change, in the concentration of the micro-organisms in a sensing region of a corresponding microfluidic channel from the oscillations in the corresponding electrical signal.

The controller may be configured to determine a change, or a rate of change, in the concentration of the micro-organisms in the sensing region of the corresponding microfluidic channel from a frequency of the oscillations in the corresponding electrical signal.

The controller may be configured to determine a change, or a rate of change, in the concentration of the micro-organisms in the sensing region of one microfluidic channel containing a first micro-organism growth-inhibiting substance relative to a change, or a rate of change, in the concentration of the micro-organisms in the sensing region of a microfluidic channel containing a different micro-organism growth-inhibiting substance based on the oscillations in the electrical signal corresponding to the microfluidic channel containing the first micro-organism growth-inhibiting substance and the oscillations in the electrical signal corresponding to the microfluidic channel containing the different micro-organism growth-inhibiting substance.

The controller may be configured to determine a change, or a rate of change, in the concentration of the micro-organisms in the sensing region of each microfluidic channel containing an micro-organism growth-inhibiting substance relative to a change, or a rate of change, in the concentration of the micro-organisms in the sensing region of the microfluidic channel which does not contain any micro-organism growth-inhibiting substance based on the oscillations in the electrical signal corresponding to each microfluidic channel containing an micro-organism growth-inhibiting substance and the oscillations in the electrical signal corresponding to the microfluidic channel which does not contain any micro-organism growth-inhibiting substance.

The optical source may comprise a coherent optical source or a single frequency optical source such as a laser or an optical parametric oscillator (OPO).

The optical source may comprise a continuous wave (CW) optical source.

The reader apparatus may comprise a heater for heating the sensor device.

The reader apparatus may comprise one or more alignment stages for aligning the optical source and/or an optical fiber-pigtail of an optical source relative to the sensor device.

The reader apparatus may comprise one or more alignment stages for aligning the one or more optical detectors relative to the sensor device.

The reader apparatus may comprise one or more alignment stages for aligning the sensor device relative to at least one of an optical source, an optical fiber-pigtail of an optical source and the one or more optical detectors.

The reader apparatus may comprise a syringe pump for injecting the fluid containing the micro-organisms into each microfluidic channel. Once the fluid is injected into the microfluidic channels, the flow of fluid is stopped. This may prevent any build-up of micro-organisms in any of the microfluidic channels which is related to the flow of fluid and which is not related to the growth of micro-organisms.

According to at least one aspect of the present disclosure there is provided a sensing system for sensing a change in a concentration of micro-organisms, the sensing system comprising the sensor device as described above and the reader apparatus as described above.

According to at least one aspect of the present disclosure there is provided a sensing method for sensing a change in a concentration of micro-organisms, the sensing method comprising:

passing a fluid containing micro-organisms along a microfluidic channel;

physically trapping micro-organisms when the fluid flows along the microfluidic channel so as to concentrate the micro-organisms in a sensing region of the microfluidic channel;

propagating sensing light along a sensing arm of a waveguide interferometer;

propagating reference light along a reference arm of the waveguide interferometer; and

interfering the sensing light with the reference light,

wherein the waveguide interferometer and the microfluidic channel are configured so that the sensing light interacts with micro-organisms in the sensing region of the microfluidic channel.

According to at least one aspect of the present disclosure there is provided a sensing method for sensing a change in a concentration of micro-organisms, the sensing method comprising:

passing a fluid containing micro-organisms along a plurality of microfluidic channels;

propagating sensing light along a sensing arm of each waveguide interferometer of a plurality of waveguide interferometers;

propagating reference light along a reference arm of each waveguide interferometer of the plurality of waveguide interferometers;

interfering the sensing light with the corresponding reference light,

wherein each waveguide interferometer and the corresponding microfluidic channel are configured so that the sensing light of each waveguide interferometer interacts with a greater concentration of the micro-organisms in the corresponding microfluidic channel than the corresponding reference light,

wherein one of the microfluidic channels contains a first micro-organism growth-inhibiting substance, and

wherein one or more of the other microfluidic channels contains a corresponding micro-organism growth-inhibiting substance which is different to the first micro-organism growth-inhibiting substance and/or one or more of the other microfluidic channels does not contain any micro-organism growth-inhibiting substance.

The micro-organisms may comprise bacteria and each micro-organism growth-inhibiting substance may comprise an antibiotic.

### BRIEF DESCRIPTION OF THE DRAWINGS

A system, method and sensor device for sensing a change in a concentration of micro-organisms will now be described by way of non-limiting example only with reference to the following drawings of which:

FIG. 1A is a schematic of a sensing system comprising a sensor device and the reader apparatus;

FIG. 1B is a schematic plan view of a photonic chip of the sensor device of FIG. 1A, with a laser and a plurality of photodetectors of the reader apparatus of FIG. 1A aligned relative to the photonic chip;

FIG. 2A is a schematic view of an underside of a lower layer of a microfluidic chip of the sensor device of FIG. 1A;

FIG. 2B is a schematic cross-section on BB of the lower layer of the microfluidic chip shown in FIG. 2A;

FIG. 2C is a schematic cross-section on AA of the lower layer of the microfluidic chip shown in FIG. 2A;

FIG. 3A is a schematic view of an underside of an upper layer of a microfluidic chip of the sensor device of FIG. 1A;

FIG. 3B is a schematic cross-section on YY of the upper layer of the microfluidic chip shown in FIG. 3A;

FIG. 4 is a schematic plan view of the lower layer of the microfluidic chip of FIG. 2A and the photonic chip of FIG. 1B (before the upper layer of the microfluidic chip of FIG. 3A is located above the lower layer of the microfluidic chip of FIG. 2A), showing the alignment between the lower layer of the microfluidic chip and the photonic chip;

FIG. 5 is a schematic plan view of a filtering arrangement of the lower layer of the microfluidic chip of FIG. 2A;

FIG. 6 is a schematic plan view of an alternative filtering arrangement for the lower layer of the microfluidic chip of FIG. 2A;

FIG. 7A is a schematic plan view of a waveguide interferometer defined on an upper side of the photonic chip of FIG. 1B and a corresponding microfluidic channel defined by the lower layer of the microfluidic chip of FIG. 2A;

FIG. 7B is a detailed schematic plan view of a trapping arrangement and a sensing region of FIG. 7A;

FIG. 7C is a schematic cross-section on XX of FIG. 7B;

FIG. 7D is a schematic cross-section on YY of FIG. 7B;

FIG. 8 is a schematic plan view of a first alternative trapping arrangement and sensing region for the sensing device of FIG. 1A;

FIG. 9 is a schematic plan view of a second alternative trapping arrangement and sensing region for the sensing device of FIG. 1A;

FIG. 10 is a schematic plan view of a third alternative trapping arrangement and sensing region for the sensing device of FIG. 1A;

FIG. 11 is a schematic plan view of a fourth alternative trapping arrangement and sensing region for the sensing device of FIG. 1A; and

FIG. 12 is a schematic plan view of a fifth alternative trapping arrangement and sensing region for the sensing device of FIG. 1A.

### DETAILED DESCRIPTION OF THE DRAWINGS

Whilst many of the features of the systems and methods are described below in specific combinations, one of ordinary skill in the art will understand that many of the features provide the same effects or advantages described below when used in combinations other than the specific combinations described, and that many of the features provide the same effects or advantages described below when the features are used in isolation from any of the other features described below.

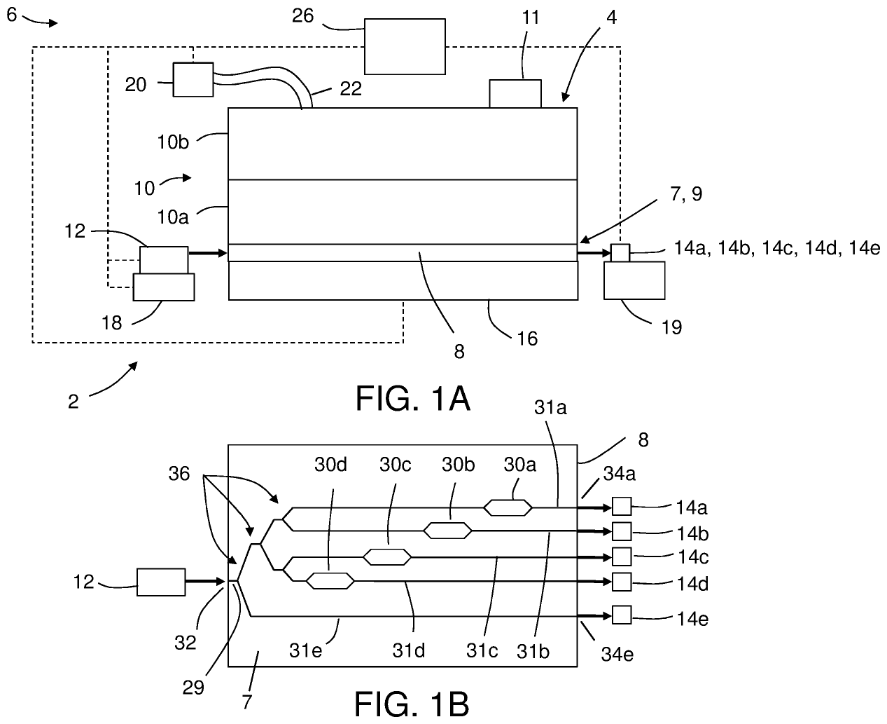

Referring initially to FIG. 1A, there is shown a sensing system generally designated 2 for sensing a change in a concentration of micro-organisms in the form of bacteria in a sample of fluid in the form of urine, and, in particular though not exclusively, for sensing the susceptibility of the bacteria to one or more antibiotics. The sensing system 2 includes a sensor device generally designated 4 and a reader apparatus generally designated 6 for reading the sensor device 4.

The sensor device 4 includes a photonic chip in the form of a disposable silicon-on-insulator photonic chip 8 and a disposable microfluidic chip 10 comprising or formed from polydimethylsiloxane (PDMS). The microfluidic chip 10 includes a lower layer 10a and an upper layer 10b. The microfluidic chip 10 also includes some absorbent material 11 for absorbing a fluid.

As will be described in more detail below, features of the photonic chip 8 are aligned with features of the microfluidic chip 10. An upper side 7 of the photonic chip 8 is then attached, for example bonded, to a lower side 9 of the microfluidic chip 10 so as to avoid any subsequent misalignment of the features of the photonic chip 8 and the features of the microfluidic chip 10.

The reader apparatus 6 includes an optical source in the form of a single frequency, continuous-wave laser 12 configured to emit light at a wavelength of 1550 nm and a plurality of optical detectors in the form of a plurality of photodiodes 14a, 14b, 14c, 14d and 14e, wherein each photodiode 14a, 14b, 14c, 14d and 14e is configured to detect light at a wavelength of 1550 nm. The reader apparatus 6 also includes one or more alignment stages 18 for aligning the laser 12 relative to the sensor device 4, one or more alignment stages 19 for aligning the photodiodes 14a, 14b, 14c, 14d and 14e relative to the sensor device 4. Although not shown explicitly in FIGS. 1A and 1B, one of skill in the art will understand that the reader apparatus 6 may include one or more lenses such as one or more objective lenses for coupling light output from the laser 12 into the photonic chip 8. The reader apparatus 6 further includes a heater 16 for heating the sensor device 4, a syringe pump 20, and a length of tubing 22 that connects the syringe pump 20 to a fluid inlet of the upper layer 10b of the microfluidic chip 10. The reader apparatus 6 also includes a controller 26. As indicated by the dashed lines in FIG. 1A, the controller 26 is configured to control the laser 12, the heater 16, the one or more alignment stages 18, 19 and the syringe pump 20, and to receive an electrical signal from each photodiode 14a, 14b, 14c, 14d and 14e.

FIG. 1B shows a plan view of the photonic chip 8 after alignment of the laser 12 and the photodiodes 14a, 14b, 14c, 14d and 14e to the sensor device 4. As shown in FIG. 1B, the photonic chip 8 defines a single-mode input waveguide 29, a plurality of waveguide splitters or Y-junctions 36, a plurality of waveguide interferometers in the form of four identical Mach-Zehnder waveguide interferometers 30a, 30b, 30c and 30d, a plurality of single-mode output waveguides 31a, 31b, 31c and 31d, and a single-mode reference waveguide 31e. The photonic chip 8 defines a single optical input 32 at an input of the input waveguide 29 and a plurality of optical outputs 34a, 34b, 34c, 34d and 34e at the output of the output waveguides 31a, 31b, 31c, 31d and the reference waveguide 31e respectively. The input waveguide 29 connects the single optical input 32 of the photonic chip 8 to an input of a first one of the waveguide splitters or Y-junctions 36. The waveguide splitters or Y-junctions 36 connect the input waveguide 29 to an input of each of the waveguide interferometers 30a, 30b, 30c and 30d and to an input end of the reference waveguide 31e. An output of each of the waveguide interferometers 30a, 30b, 30c and 30d is connected to a corresponding one of optical outputs 34a, 34b, 34c, and 34d via a corresponding one of the output waveguides 31a, 31b, 31c and 31d respectively.

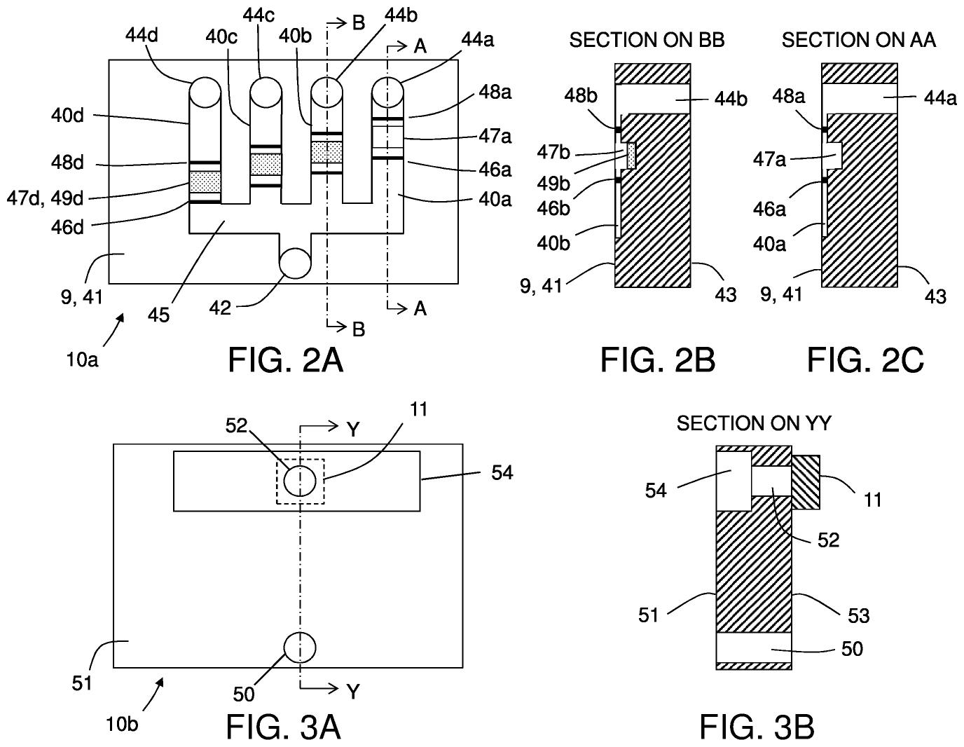

As shown in FIGS. 2A-2C, an underside 41 of the lower layer 10a of the microfluidic chip 10 defines a plurality of microfluidic channels in the form of four microfluidic channels 40a, 40b, 40c and 40d. The lower layer 10a of the microfluidic chip 10 further defines a fluid inlet in the form of a through-hole 42 and a plurality of fluid outlets in the form of a plurality of through-holes 44a, 44b, 44c and 44d. The fluid inlet 42 and each of the fluid outlets 44a, 44b, 44c and 44d extends from the underside of the lower layer 10a to an upper side 43 of the lower layer 10a. The lower layer 10a of the microfluidic chip 10 also defines a fluid manifold 45 connected to the fluid inlet 42. Each microfluidic channel 40a, 40b, 40c and 40d extends from the fluid manifold 45 to the corresponding fluid outlet 44a, 44b, 44c and 44d respectively. The lower layer 10a of the microfluidic chip 10 further defines a filtering arrangement 46a, 46b, 46c and 46d for trapping debris or particulates which are greater in size than the bacteria, a well in the form of a recess 47a, 47b, 47c and 47d located downstream of the filtering arrangement 46a, 46b, 46c and 46d, and a trapping arrangement 48a, 48b, 48c and 48d for trapping bacteria located downstream of the recess 47a, 47b, 47c and 47d in each of the microfluidic channels 40a, 40b, 40c and 40d respectively. As indicated in FIG. 2A, microfluidic channels 40b, 40c and 40d contain different micro-organism growth-inhibiting substances in the form of different antibiotics in the corresponding recess 47b, 47c and 47d. Specifically, each recess 47b, 47c, 47d contains a corresponding different dried antibiotic formed by dispensing (e.g. pipetting) the antibiotic concerned in solution into the recess 47b, 47c, 47d and leaving the solution to dry. The recess 47a of microfluidic channel 40a does not contain any antibiotic.

As shown in FIGS. 3A and 3B, the upper layer 10b of the microfluidic chip 10 defines a fluid inlet in the form of a through-hole 50 and a fluid outlet in the form of a further through-hole 52. An underside 51 of the upper layer 10b of the microfluidic chip 10 defines a recess 54 which may serve as a fluid collection reservoir. The fluid outlet 52 extends from the recess 54 on the underside 51 of the upper layer 10b to an upper side 53 of the upper layer 10b. When the upper layer 10b of the microfluidic chip 10 is aligned with the lower layer 10a of the microfluidic chip 10 with the underside 51 of the upper layer 10b disposed towards the upper side 43 of the lower layer 10a, the fluid inlets 50 and 42 of the upper and lower layers 10b and 10a respectively are aligned and the recess 54 defined in the underside 51 of the upper layer 10b is aligned with the fluid outlets 44a, 44b, 44c and 44d at the upper side 43 of the lower layer 10a so as to permit the recess 54 to receive fluid from each of the fluid outlets 44a, 44b, 44c and 44d. As will be described in more detail below, the syringe pump 20 is used to inject fluid containing bacteria into the fluid inlets 50 and 42, along the microfluidic channels 40a, 40b, 40c and 40d and out through the fluid outlets 44a, 44b, 44c and 44d, into the recess 54 and then from the recess 54 to the fluid outlet 52 whereupon any excess fluid is absorbed by the absorbent material 11.

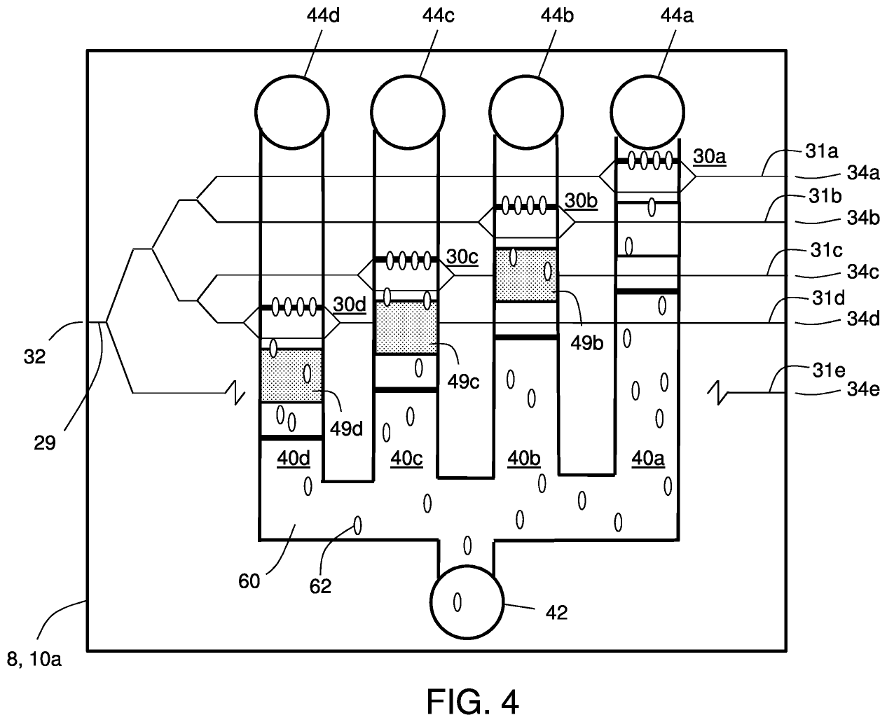

Referring now to FIG. 4, there is shown a plan view showing the alignment of the lower layer 10a of the microfluidic chip 10 with the photonic chip 8 after a fluid 60 containing bacteria 62 has been injected into each of the microfluidic channels 40a, 40b, 40c and 40d via the fluid inlet 42. As shown in FIG. 4, the trapping arrangement 48a, 48b, 48c and 48d of each microfluidic channel 40a, 40b, 40c and 40d is generally aligned with a corresponding sensing arm of a corresponding one of the waveguide interferometers 30a, 30b, 30c and 30d respectively.

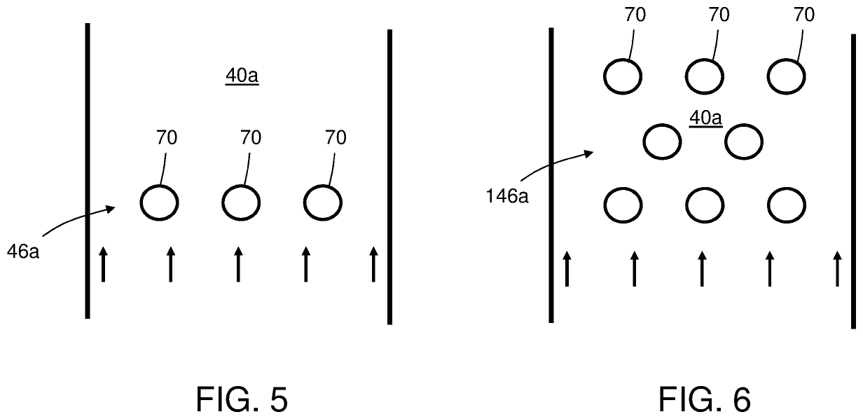

Referring now to FIG. 5, there is shown a plan view of a region of microfluidic channel 40a in the vicinity of the corresponding filtering arrangement 46a. As shown in FIG. 4, the filtering arrangement 46a includes a plurality of pillars 70 extending into the microfluidic channel 40a defining a plurality of gaps, wherein each gap is greater in size than the bacteria 62 contained in the fluid 60. Specifically, each gap is larger than the maximum dimension of the bacteria 62. One of ordinary skill in the art will understand that the other filtering arrangements 46b, 46c and 46d in the other microfluidic channels 40b, 40c and 40d are identical to filter arrangement 46a.

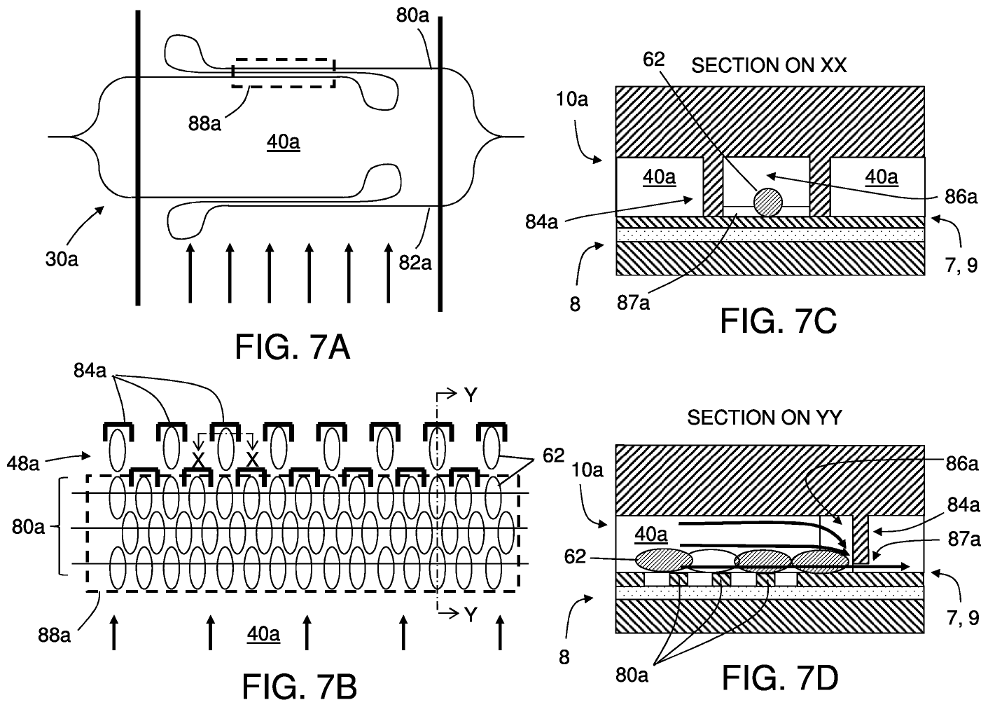

Referring now to FIG. 7A, there is shown a detailed view of waveguide interferometer 30a and a corresponding region of microfluidic channel 40a. As shown in FIG. 7A, waveguide interferometer 30a includes a single-mode waveguide sensing arm 80a and a single-mode waveguide reference arm 82a. A section of the sensing arm 80a is folded so as to define three parallel waveguide portions to increase the interaction length between light in the sensing arm 80a and the bacteria 62 in the fluid 60 in the microfluidic channel 40a. Similarly, a section of the reference arm 82a is folded so as to define three parallel waveguide portions. Although not shown explicitly in FIG. 7A, one of skill in the art will understand that the sensing and reference arms 80a and 82a are unbalanced (i.e. the sensing and reference arms 80a and 82a have different optical lengths) for improved measurement sensitivity.

The trapping arrangement 48a comprises two staggered rows of traps 84a located adjacent to, and downstream from, the folded section of the sensing arm 80a in the flow of fluid 60. Each trap 84a is configured to physically trap the bacteria 62 when the fluid 60 flows along the microfluidic channel 40a. Specifically, as shown in FIGS. 7C and 7D, each trap 84a comprises one or more features extending into the microfluidic channel 40a so as to define a bay 86a in the microfluidic channel 40a for accommodating one or more bacteria 62. Each trap 84a also defines a gap 87a between the trap 84a and the upper surface 7 of the photonic chip 8, which gap 87a is configured to allow fluid 60 in the microfluidic channel 40a to flow under the trap 84a but to prevent bacteria 62 from passing under the trap 84a. Without wishing to be bound by theory, it may be the case that the flow of fluid over the bacteria 62 trapped in the trap 84a and through the gap 87a between the trap 84a and the upper surface 7 of the photonic chip 8 may cause the fluid 60 to exert a downward force on the bacteria 62 trapped in the trap 84a, which downward force may serve to pin the bacteria 62 trapped in the trap 84a onto the upper surface 7 of the photonic chip 8. As will be appreciated from FIGS. 7B, 7C and 7D, in use, the trapping arrangement 48a serves to concentrate bacteria 62 in a sensing region 88a of the microfluidic channel 40a, which sensing region 88a is located above the folded section of the sensing arm 80a.

One of ordinary skill in the art will understand that the waveguide interferometers 30b, 30c and 30d are identical to the waveguide interferometer 30a described with reference to FIG. 7A. Specifically, each waveguide interferometer 30b, 30c and 30d includes a sensing arm 80b, 80c and 80d and a reference arm 82b, 82c and 82d respectively. Each sensing arm 80b, 80c and 80d has a corresponding folded section. Similarly, each reference arm 82b, 82c and 82d has a corresponding folded section. Each of the other microfluidic channels 40b, 40c and 40d defines a corresponding trapping arrangement 48b, 48c and 48d respectively. Each of the trapping arrangements 48b, 48c and 48d are identical to the trapping arrangement 48a described with reference to FIGS. 7B, 7C and 70 and serve to concentrate bacteria 62 in a corresponding sensing region 88b, 88c and 88d respectively located above the folded section of the corresponding sensing arm 80b, 80c and 80d respectively.

In use, a urine sample (<1 ml is required) is combined with biological growth media powder (here Mueller Hinton Broth at 21 mg/ml concentration, though others could be used) in a tube and inverted several times. The media powder is used to promote bacterial growth in urine and to buffer out chemical and pH differences between urine samples. The urine/media solution 60, 62 is then drawn into a syringe and connected to the microfluidic chip 10 via a flat syringe needle and the flexible tubing 22 which is connected to the fluid inlet 50 of upper layer 10b of the microfluidic chip 10. The syringe pump 20 pumps the urine/media solution 60, 62 into the microfluidic channels 40a, 40b, 40c and 40d of the microfluidic chip 10, before stopping the flow.

As the urine/media solution 60, 62 enters the microfluidic channels 40a, 40b, 40c and 40d, the filtering arrangements 46a, 46b, 46c and 46d trap any debris which is larger than the bacteria 62 within the urine/media solution 60, 62, the dried antibiotics 49b, 49c and 49d are re-constituted in the urine/media solution 60, 62 in the microfluidic channels 40b, 40c and 40d, and any bacteria 62 present in the urine/media solution 60, 62 are physically trapped via the trapping arrangements 48a, 48b, 48c and 48d in the sensing regions 88a, 88b, 88c and 88d above the sensing arm 80a, 80b, 80c and 80d of the corresponding waveguide interferometer 30a, 30b, 30c and 30d respectively. Any non-bacterial material that makes it to the trapping arrangements 48a, 48b, 48c and 48d may cause an initial change in intensity of the light at the outputs of the waveguide interferometers 30a, 30b, 30c and 30d during fluid flow, but will not contribute to the dynamic change in intensity of the light at the outputs of the waveguide interferometers 30a, 30b, 30c and 30d that results from bacterial growth over time.

In use, one of ordinary skill in the art will understand that the sensor device 4 may have a standard form factor and the reader apparatus 6 may include one or more reference features relative to which the sensor device 4 may be aligned to achieve an initial coarse alignment between the sensor device 4 and the reader apparatus 6. The controller 26 of the reader apparatus 6 then controls the alignment stages 18, 19 so as to actively align the laser 12 to the sensor device 4 and so as to actively align the plurality of photodiodes 14a, 14b, 14c, 14d and 14e to the sensor device 4. Specifically, with the laser 12 emitting single-frequency continuous-wave (CW) light at 1550 nm, the controller 26 controls the alignment stages 18, 19 so as to maximize the value of the electrical signal generated by the photodiode 14e corresponding to the reference waveguide 31.

The heater 16 maintains the temperature of the sensor device 4 at the optimum growth temperature of 37° C. and bacteria trapped in the sensing regions 88a, 88b, 88c and 88d grow in the urine/media powder mixture. Bacterial growth alters the effective refractive index of the sensing arm 80a, 80b, 80c and 80d of each waveguide interferometer 30a, 30b, 30c and 30d respectively, thus inducing an optical phase change relative to the corresponding reference arm 82a, 82b, 82c and 82d respectively. Light from the laser 12 passes through each of the waveguide interferometers 30a, 30b, 30c and 30d and is measured by the corresponding photodetectors 14a, 14b, 14c and 14d respectively.

The laser 12 emits light with a Transverse Magnetic (TM) polarization, because it has been found that this polarization provides the greatest measurement sensitivity and better fabrication tolerances. As the optical phase on the sensing arm 80a, 80b, 80c, 80d changes due to bacterial growth, this induces an intensity change at the waveguide output of each waveguide interferometer 30a, 30b, 30c and 30d respectively due to interference between light propagating in each sensing arm 80a, 80b, 80c, 80d with light propagating in the corresponding reference arm 82a, 82b, 82c, 82d.

Although the reference arms 82a, 82b, 82c and 82d of each waveguide interferometer 30a, 30b, 30c and 30d are exposed to the fluid 60 and the bacteria 62 so that light propagating along the reference arms 82a, 82b, 82c and 82d can interact with the fluid 60 and the bacteria 62 in the corresponding microfluidic channels 40a, 40b, 40c and 40d, due to the absence of any trapping arrangements in the reference arms 82a, 82b, 82c and 82d, the concentration of bacteria in the vicinity of the reference arms 82a, 82b, 82c and 82d is much lower than the concentration of bacteria in the vicinity of the sensing regions of the corresponding sensing arms 80a, 80b, 80c and 80d. Exposing both the sensing arms 80a, 80b, 80c and 80d and the reference arms 82a, 82b, 82c and 82d of each waveguide interferometer 30a, 30b, 30c and 30d to the fluid 60 and the bacteria 62 in this way helps to improve measurement immunity to any changes in the bulk refractive index of the fluid and the bacteria that are not due to bacterial growth.

If the bacteria 62 in any of the sensing regions 88a, 88b, 88c and 88d grow, the phase and thus the intensity at the output of the corresponding waveguide interferometer 30a, 30b, 30c and 30d changes over time. The intensity, as measured by the photodiodes 14a, 14b, 14c and 14d, traces out a series of fringes or oscillations corresponding to the optical interference pattern as the bacteria grow. The frequency of these fringes is dependent on the amount and rate of growth of bacteria. If the bacteria are susceptible to the antibiotic 49b, 49c, 49d that was re-constituted in the corresponding microfluidic channel 40b, 40c, 40d, the bacteria in the microfluidic channel 40b, 40c, 40d concerned stop growing and the rate of intensity change associated with the microfluidic channel 40b, 40c, 40d concerned reduces relative to the rate of intensity change associated with the reference microfluidic channel 40a which does not contain any antibiotic. In effect, this results in the intensity fringes or oscillations reducing in frequency and/or flattening out as the phase shift slows or stops in the sensing arm 80b, 80c, 80d of the waveguide interferometer 30b, 30c, 30d which corresponds to the microfluidic channel 40b, 40c, 40d concerned.

The sensor device 4 is designed such that multiple antibiotics 49b, 49c, 49d can be tested at the same time, in addition to the reference microfluidic channel 40a without antibiotics. The controller 26 of the reader apparatus 6 can then determine the most effective or appropriate antibiotic from the electrical signals generated by the photodetectors 14a, 14b, 14c and 14d. Specifically, the controller 26 compares the frequency, size and/or shape of the fringes or oscillations in the electrical signals corresponding to the microfluidic channels 40b, 40c and 40d relative to the frequency, size and/or shape of the fringes or oscillations in the electrical signal corresponding to the reference microfluidic channel 40a without antibiotic, and to each other. The controller 26 identifies the most effective or appropriate antibiotic as the antibiotic in the microfluidic channel corresponding to the electrical signal with the lowest oscillation frequency. One of ordinary skill in the art will understand that the use of such a sensor device 4 having waveguide interferometers 30a, 30b, 30c and 30d to measure the relative growth or decline of bacteria in the presence of one or more different antibiotics as described above, does not require the reader apparatus 6 to have a spectrometer or a tuneable light source, thereby allowing rapid measurements of the efficacy of different bacteria to be performed using a relatively simple disposable sensor device 4 and a relatively simple reader apparatus 6.

Referring to FIG. 6, there is shown an alternative filtering arrangement 146a for use in microfluidic channel 40a in place of the filtering arrangement 46a described with reference to FIG. 5. As shown in FIG. 6, the alternative filtering arrangement 146a includes a plurality of staggered rows of pillars 70 extending into the microfluidic channel 40a defining a plurality of gaps, wherein each gap is greater in size than the bacteria 62 contained in the fluid 60. Specifically, each gap is larger than the maximum dimension of the bacteria 62. One of ordinary skill in the art will understand that the other microfluidic channels 40b, 40c and 40d may have alternative filtering arrangements which are identical to the alternative filter arrangement 146a.

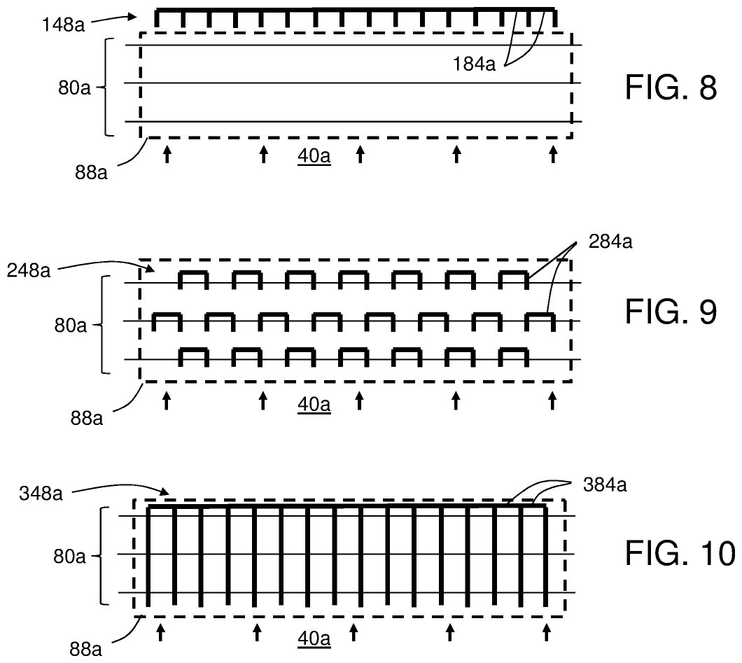

Referring to FIG. 8, there is shown a first alternative trapping arrangement 148a for use in microfluidic channel 40a in place of the trapping arrangement 48a described with reference to FIGS. 7B, 7C and 7D. As shown in FIG. 8, the first alternative trapping arrangement 148a defines a plurality of traps 184a located adjacent to, and downstream from, the folded section of the sensing arm 80a in the flow of fluid 60. Each trap 184a is configured to physically trap the bacteria 62 when the fluid 60 flows along the microfluidic channel 40a. Each trap 184a comprises one or more features extending into the microfluidic channel 40a so as to define a corresponding bay in the microfluidic channel 40a for accommodating one or more bacteria 62. Each trap 184a also defines a gap between the trap 84a and the upper surface 7 of the photonic chip 8, which gap is configured to allow fluid 60 in the microfluidic channel 40a to flow through the gap under the trap 184a but to prevent bacteria 62 from passing through the gap under the trap 184a. In use, the trapping arrangement 148a serves to concentrate bacteria 62 in the sensing region 88a of the microfluidic channel 40a, which sensing region 88a is located above the folded section of the sensing arm 80a. Corresponding trapping arrangements may be provided in the other microfluidic channels 40b, 40c and 40d which are identical to the trapping arrangement 148a so as to concentrate bacteria 62 in the corresponding sensing regions 88b, 88c and 88d of the other microfluidic channels 40b, 40c and 40d.

Referring to FIG. 9, there is shown a second alternative trapping arrangement 248a for use in microfluidic channel 40a in place of the trapping arrangement 48a described with reference to FIGS. 7B, 7C and 7D. As shown in FIG. 9, the second alternative trapping arrangement 248a defines three staggered rows of traps 284a which are aligned with the folded section of the sensing arm 80a. Specifically, each row of traps 284a is generally aligned with one of the waveguide portions in the folded section of the sensing arm 80a. Each trap 284a is configured to physically trap the bacteria 62 when the fluid 60 flows along the microfluidic channel 40a. Each trap 284a comprises one or more features extending into the microfluidic channel 40a so as to define a corresponding bay in the microfluidic channel 40a for accommodating one or more bacteria 62. Each trap 284a also defines a gap between the trap 284a and the upper surface 7 of the photonic chip 8, which gap is configured to allow fluid 60 in the microfluidic channel 40a to flow through the gap under the trap 284a but to prevent bacteria 62 from passing through the gap under the trap 284a. In use, the trapping arrangement 248a serves to concentrate bacteria 62 in the sensing region 88a of the microfluidic channel 40a, which sensing region 88a is located above the folded section of the sensing arm 80a. Corresponding trapping arrangements may be provided in the other microfluidic channels 40b, 40c and 40d which are identical to the trapping arrangement 248a so as to concentrate bacteria 62 in the corresponding sensing regions 88b, 88c and 88d of the other microfluidic channels 40b, 40c and 40d.

Referring to FIG. 10, there is shown a third alternative trapping arrangement 348a for use in microfluidic channel 40a in place of the trapping arrangement 48a described with reference to FIGS. 7B, 7C and 7D. As shown in FIG. 10, the third alternative trapping arrangement 348a defines a single row of traps 384a which are aligned with the folded section of the sensing arm 80a. Specifically, each trap 384a extends in the direction of fluid flow across all three waveguide portions in the folded section of the sensing arm 80a. Each trap 384a is configured to physically trap the bacteria 62 when the fluid 60 flows along the microfluidic channel 40a. Each trap 384a comprises one or more features extending into the microfluidic channel 40a so as to define a corresponding bay in the microfluidic channel 40a for accommodating one or more bacteria 62. Each trap 384a also defines a gap between the trap 384a and the upper surface 7 of the photonic chip 8, which gap is configured to allow fluid 60 in the microfluidic channel 40a to flow through the gap under the trap 384a but to prevent bacteria 62 from passing through the gap under the trap 384a. In use, the trapping arrangement 348a serves to concentrate bacteria 62 in the sensing region 88a of the microfluidic channel 40a, which sensing region 88a is located above the folded section of the sensing arm 80a. Corresponding trapping arrangements may be provided in the other microfluidic channels 40b, 40c and 40d which are identical to the trapping arrangement 348a so as to concentrate bacteria 62 in the corresponding sensing regions 88b, 88c and 88d of the other microfluidic channels 40b, 40c and 40d.

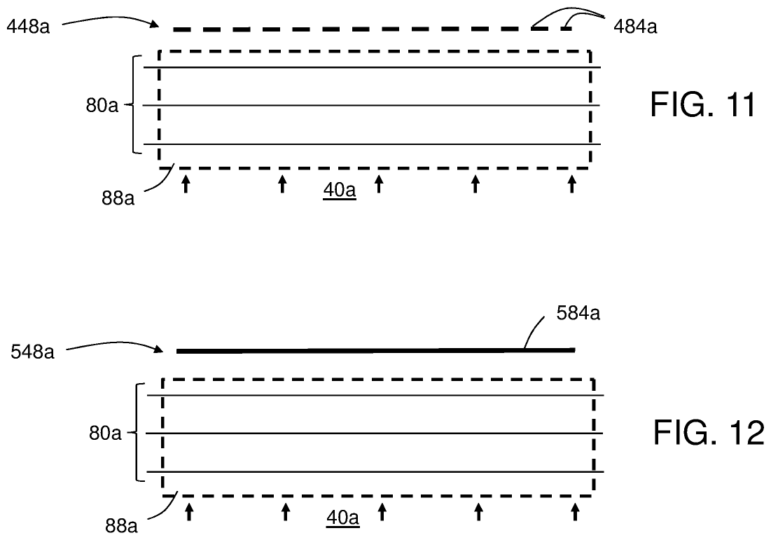

Referring to FIG. 11, there is shown a fourth alternative trapping arrangement 448a for use in microfluidic channel 40a in place of the trapping arrangement 48a described with reference to FIGS. 7B, 7C and 7D. As shown in FIG. 11, the fourth alternative trapping arrangement 448a defines a row of trapping features 484a which are located adjacent to, and downstream from, the folded section of the sensing arm 80a in the flow of fluid 60. Adjacent trapping features 484a define a gap therebetween which is configured to allow fluid 60 in the microfluidic channel 40a to flow through the gap but to prevent bacteria 62 from passing through the gap. Each trapping feature 484a also defines a gap between the trapping feature 484a and the upper surface 7 of the photonic chip 8, which gap is configured to allow fluid 60 in the microfluidic channel 40a to flow through the gap under the trapping feature 484a but to prevent bacteria 62 from passing through the gap under the trapping feature 484a. In use, the trapping arrangement 448a serves to concentrate bacteria 62 in the sensing region 88a of the microfluidic channel 40a, which sensing region 88a is located above the folded section of the sensing arm 80a. Corresponding trapping arrangements may be provided in the other microfluidic channels 40b, 40c and 40d which are identical to the trapping arrangement 448a so as to concentrate bacteria 62 in the corresponding sensing regions 88b, 88c and 88d of the other microfluidic channels 40b, 40c and 40d.

Referring to FIG. 12, there is shown a fifth alternative trapping arrangement 548a for use in microfluidic channel 40a in place of the trapping arrangement 48a described with reference to FIGS. 7B, 7C and 7D. As shown in FIG. 12, the fifth alternative trapping arrangement 548a defines a continuous trapping feature 584a which is located adjacent to, and downstream from, the folded section of the sensing arm 80a in the flow of fluid 60. The trapping feature 584a defines a gap between the trapping feature 584a and the upper surface 7 of the photonic chip 8, which gap is configured to allow fluid 60 in the microfluidic channel 40a to flow through the gap under the trapping feature 584a but to prevent bacteria 62 from passing through the gap under the trapping feature 584a. In use, the trapping arrangement 548a serves to concentrate bacteria 62 in the sensing region 88a of the microfluidic channel 40a, which sensing region 88a is located above the folded section of the sensing arm 80a. Corresponding trapping arrangements may be provided in the other microfluidic channels 40b, 40c and 40d which are identical to the trapping arrangement 548a so as to concentrate bacteria 62 in the corresponding sensing regions 88b, 88c and 88d of the other microfluidic channels 40b, 40c and 40d.

One of ordinary skill in the art will understand that various modifications are possible to the system and methods described above. For example, rather than dispensing the antibiotics 49b, 49c and 49d in solution into the corresponding wells or recesses 47b, 47c and 47d in the corresponding microfluidic channels 40b, 40c and 40d using a pipette, one or more of the antibiotics 49b, 49c and 49d may be dispensed by inkjet printing and left to dry.

Although the reference arms 82a, 82b, 82c and 82d of each waveguide interferometer 30a, 30b, 30c and 30d are exposed to the fluid 60 and the bacteria 62 so that light propagating along the reference arms 82a, 82b, 82c and 82d can interact with the fluid 60 and the bacteria 62 in the corresponding microfluidic channels 40a, 40b, 40c and 40d, the sensing device 4 may be configured to prevent exposure of the reference arms 82a, 82b, 82c and 82d of each waveguide interferometer 30a, 30b, 30c and 30d to the fluid 60 and the bacteria 62 so as to prevent light propagating along the reference arms 82a, 82b, 82c and 82d from interacting with the fluid 60 and the bacteria 62 in the corresponding microfluidic channels 40a, 40b, 40c and 40d. For example, the sensor device 4 may include a cover layer or mask which prevents exposure of the reference arms 82a, 82b, 82c and 82d to the fluid 60 and the bacteria 62 so as to prevent light propagating along the reference arms 82a, 82b, 82c and 82d from interacting with the fluid 60 and the bacteria 62 in the corresponding microfluidic channels 40a, 40b, 40c and 40d, whilst still exposing of the sensing arms 80a, 80b, 80c and 80d to the fluid 60 containing the bacteria 62 so as to allow light propagating along the sensing arms 80a, 80b, 80c and 80d to interact with the fluid 60 and the bacteria 62 in the corresponding microfluidic channels 40a, 40b, 40c and 40d. Preventing exposure of the reference arms 82a, 82b, 82c and 82d of each waveguide interferometer 30a, 30b, 30c and 30d to the fluid 60 and the bacteria 62 in this way may enhance the measurement sensitivity, but may reduce the measurement immunity to any changes in the bulk refractive index of the fluid and the bacteria which do not arise from growth of the bacteria.

Although the photonic chip 8 is defined using a silicon-on-insulator material system, the photonic chip 8 may comprise, or be formed from, a photonic material system including, though not exclusively limited to, silica or glass, polymer, silicon nitride and the like.

Although the photonic chip 8 was described above as defining waveguide splitters or Y-junctions for connecting the single optical input 32 to an input of each of the waveguide interferometers 30a, 30b, 30c and 30d, the photonic chip 8 may instead define directional couplers for connecting the single optical input 32 to an input of each of the waveguide interferometers 30a, 30b, 30c and 30d. Alternatively, the photonic chip 8 may define a multi-mode interference (MMI) splitter for connecting the single optical input 32 to an input of each of the waveguide interferometers 30a, 30b, 30c and 30d. A MMI splitter may be more compact than the use of splitters, Y-junctions or directional couplers.

The photonic chip 8 may define a mode converter or a spot-size converter for converting an optical field of the light incident on the photonic chip 8 into an optical field having a mode profile which more closely matches a mode profile associated with the waveguide splitters and the waveguide interferometers.

The photonic chip 8 may define a grating input coupler for coupling light from the laser into the photonic chip 8. The photonic chip 8 may define one or more grating output couplers for coupling light from the photonic chip 8 to the photodiodes 14a, 14b, 14c, 14d and 14e.

Although the photonic chip 8 is described above as having a single optical input 32 located at a first edge of the photonic chip 8 and a plurality of optical outputs 34a, 34b, 34c, 34d and 34e located at a second edge of the photonic chip 8 opposite to the first edge, the optical input 32 and the plurality of optical outputs 34a, 34b, 34c, 34d and 34e may be located at the same edge of the photonic chip 8 and the photonic chip 8 may define at least one of the input waveguide 29, the output waveguides 31a, 31b, 31c, 31d and the reference waveguide 31e accordingly. For example, the photonic chip 8 may define at least one bend in at least one of the input waveguide 29, the output waveguides 31a, 31b, 31c, 31d and the reference waveguide 31e so that the optical input 32 and the plurality of optical outputs 34a, 34b, 34c, 34d and 34e are located at the same edge of the photonic chip 8. Such a chip arrangement may allow the laser 12 and the photodiodes 14a, 14b, 14c, 14d and 14e to be mounted on the same set of alignment stages. This may reduce the number of alignment stages needed and/or simplify alignment between the reader apparatus 6 and the photonic chip 8. In an alternative variant, the photonic chip 8 may be mounted on a set of alignment stages and the photonic chip 8 moved relative to the laser 12 and the photodiodes 14a, 14b, 14c, 14d and 14e.

Although the sensing arms 80a, 80b, 80c, 80d and the corresponding reference arms 82a, 82b, 82c, 82d of each waveguide interferometer 30a, 30b, 30c and 30d are described above as being unbalanced (i.e. having different optical lengths) for improved measurement sensitivity, one of skill in the art will understand that the sensing arms 80a, 80b, 80c, 80d and the corresponding reference arms 82a, 82b, 82c, 82d of each waveguide interferometer 30a, 30b, 30c and 30d may be balanced (i.e. have the same optical length). The use of such balanced sensing and reference arms may be better in terms of thermal stability i.e. the use of balanced sensing and reference arms may reduce any change in the intensity of the light at the output of the waveguide interferometer as a result of a change in temperature. The use of balanced sensing and reference arms may also help to cancel out any refractive index changes that are not due to a change in concentration of bacteria in the sensing region. For example, when the waveguide interferometer and the microfluidic channel are configured to allow the reference light to interact with the fluid and the bacteria in the microfluidic channel, the use of balanced sensing and reference arms may also help to cancel out any refractive index change of the fluid that is not caused by a change in concentration of bacteria in the sensing region.

The laser 12 may include one or more lenses for collimating light output from the laser 12.

The reader apparatus 6 may comprise one or more lenses, such as one or more objective lenses, for coupling light output from the laser 12 to the single optical input 32 of the photonic chip 8.

The laser 12 may include a housing or body and an output fiber-pigtail extending from the housing or body. The one or more alignment stages may be configured to move the output fiber-pigtail relative to the photonic chip without moving the housing or body of the laser.

The fiber pigtail may include, or be formed from, polarization maintaining (PM) fiber. Use of PM fiber may allow the polarization of the light coupled into the photonic chip 8 to be controlled.

The reader apparatus 6 may include a fiber collimator arrangement for collimating light output from the fiber pigtail and a lens such as an objective lens for focusing light output from the fiber collimator arrangement into an input waveguide of the photonic chip 8.

The reader apparatus 6 may include a polarizer located between the fiber collimator arrangement and the lens for polarizing, or further polarizing, the light output from the fiber collimator arrangement.

Although the laser 12 emits single-frequency continuous-wave light at a wavelength of 1550 nm, the single-frequency continuous-wave light may have any other suitable wavelength.

Although a laser 12 is used to emit single-frequency continuous-wave light, any optical source which is capable of emitting coherent CW light may be used. For example, an optical parametric oscillator (OPO) may be used.

Although the microfluidic chip 10 is defined using PDMS, the microfluidic chip 10 may comprise, or be formed from, silica or glass, polymer, silicon, silicon nitride and the like.

Rather than the lower layer 10a of the microfluidic chip 10 defining a well in the form of a recess 47a, 47b, 47c, 47d for containing an antibiotic between the filtering arrangement 46a, 46b, 46c, 46d and trapping arrangement 48a, 48b, 48c, 48d in each of the microfluidic channels 40a, 40b, 40c, 40d respectively as shown in FIGS. 2A, 2B and 2C, the lower layer 10a of the microfluidic chip 10 may define a well in the form of a through-hole for loading an antibiotic, wherein the through-hole extends through the lower layer 10a of the microfluidic chip 10 at a position between the filtering arrangement 46a, 46b, 46c, 46d and trapping arrangement 48a, 48b, 48c, 48d in each of the microfluidic channels 40a, 40b, 40c, 40d respectively. Once the lower layer 10a of the microfluidic chip 10 is bonded to the photonic chip 8, antibiotics 49b, 49c, 49d may be loaded into one or more of microfluidic channels 40b, 40c, 40d via such through-holes and the through-holes may be sealed when the upper layer 10b of the microfluidic chip 10 is subsequently placed on top of the lower layer 10a of the microfluidic chip 10. The use of such through-holes for loading an antibiotic avoids any requirement to load any antibiotics into recesses 47b, 47c, 47d in the lower layer 10a of the microfluidic chip 10 before the lower layer 10a of the microfluidic chip 10 is bonded to the photonic chip 8. This may be advantageous as it may avoid any changes, damage and/or contamination of the antibiotics 49b, 49c, 49d which may otherwise occur if the antibiotics 49b, 49c, 49d are loaded into recesses 47b, 47c, 47d in the lower layer 10a of the microfluidic chip 10 before the lower layer 10a of the microfluidic chip 10 is bonded to the photonic chip 8.

Antibiotics 49b, 49c, 49d may be introduced into each well as a fluid. Antibiotics 49b, 49c, 49d may be introduced before and/or during a measurement of bacterial growth. The microfluidic chip 10 may be configured so that each well may receive a corresponding one of the antibiotics 49b, 49c, 49d from a respective receptacle, tube or reservoir of antibiotic 49b, 49c, 49d. For example, the microfluidic chip 10 may define a separate fluid inlet for each antibiotic 49b, 49c, 49d to allow each antibiotic 49b, 49c, 49d to be injected or dispensed as a fluid into the corresponding microfluidic channel 40b, 40c, 40d respectively.

Rather than using an upper layer 10b of the microfluidic chip 10 which defines the through-hole 50 and aligning the upper layer 10b of the microfluidic chip 10 with the lower layer 10a of the microfluidic chip 10 so that the through-hole 50 is aligned with the fluid inlet 42 defined by the lower layer 10a of the microfluidic chip 10, the upper layer 10b of the microfluidic chip 10 may have a different size and/or shape to the lower layer 10a of the microfluidic chip 10 such that when the upper layer 10b of the microfluidic chip 10 is aligned with the lower layer 10a of the microfluidic chip 10, the upper layer 10b of the microfluidic chip 10 does not extend across the fluid inlet 42 defined by the lower layer 10a of the microfluidic chip 10.

The fluid reservoir 54 may be sufficiently large so as to accommodate a limited excess volume of fluid 60 when the fluid 60 and the bacteria 62 is injected into the microfluidic channels 40a, 40b, 40c and 40d of the microfluidic chip 10. This may avoid any requirement for the use of the absorbent material 11.

The lower layer 10a of the microfluidic chip 10 may be configured so that each filtering arrangement 46a, 46b, 46c, 46d is located the same distance from the fluid inlet 42. This means that the fluid 60 and the bacteria 62 should reach the filtering arrangement 46a, 46b, 46c, 46d in each microfluidic channel 40a, 40b, 40c, 40d at the same time when the fluid 60 and the bacteria 62 are injected into the microfluidic channels 40a, 40b, 40c, 40d via the fluid inlet 42.

The lower layer 10a of the microfluidic chip 10 may be configured so that each recess or well 47a, 47b, 47c, 47d is located the same distance from the fluid inlet 42. This means that the fluid 60 and the bacteria 62 should reach the recess or well 47a, 47b, 47c, 47d in each microfluidic channel 40a, 40b, 40c, 40d at the same time when the fluid 60 and the bacteria 62 are injected into the microfluidic channels 40a, 40b, 40c, 40d via the fluid inlet 42.

The lower layer 10a of the microfluidic chip 10 may be configured so that each trapping arrangement 48a, 48b, 48c, 48d is located the same distance from the fluid inlet 42. This means that the fluid 60 and the bacteria 62 should reach the trapping arrangement 48a, 48b, 48c, 48d in each microfluidic channel 40a, 40b, 40c, 40d at the same time when the fluid 60 and the bacteria 62 are injected into the microfluidic channels 40a, 40b, 40c, 40d via the fluid inlet 42.

The photonic chip 8 and the lower layer 10a of the microfluidic chip 10 may be configured so that the sensing arm 80a, 80b, 80c, 80d of each waveguide interferometer 30a, 30b, 30c, 30d is aligned relative to the corresponding trapping arrangement 48a, 48b, 48c, 48d so that sensing light in the sensing arm 80a, 80b, 80c, 80d can interact with the fluid 60 and the bacteria 62 in the corresponding sensing region 88a, 88b, 88c, 88d of the microfluidic channel 40a, 40b, 40c, 40d respectively.

Rather than combining a fluid sample with biological growth media powder before injecting the fluid sample and the media powder into the microfluidic chip 10, the media powder may be dried or formed on the microfluidic chip 10 in the same manner as the antibiotics, for example together with the antibiotics 49b, 49c, 49d at the corresponding recess or well 47b, 47c, 47d, or at the fluid inlet 42.

A biological growth media powder other than Mueller Hinton Broth may be used to promote bacterial growth. For example, L-broth biological growth media powder may be used.

Although the sensing system, method and sensor device are described above in the context of measuring the susceptibility of bacteria in a urine sample to different antibiotics, the sensing system, method and sensor device may be used to measure the susceptibility of bacteria in any bodily fluid to different antibiotics. For example, the sensing system, method and sensor device may be used to measure the susceptibility of bacteria in blood, saliva, sputum or the like to different antibiotics.

Although the sensing system, method and sensor device are described above in the context of measuring the susceptibility of bacteria in a urine sample to different antibiotics, the sensing system, method and sensor device may be used to measure the susceptibility of any micro-organism in any fluid to different micro-organism growth-inhibiting substances. For example, the sensing system, method and sensor device may be used to measure the susceptibility of fungi or algae in any fluid to different micro-organism growth-inhibiting substances.

1. A sensor device for use in sensing a change in a concentration of micro-organisms, the sensor device comprising:
a waveguide interferometer having a sensing arm and a reference arm;
a microfluidic channel for a fluid containing the micro-organisms; and
a trapping arrangement in the microfluidic channel for physically trapping the micro-organisms when the fluid flows along the microfluidic channel so as to concentrate the micro-organisms in a sensing region of the microfluidic channel,
wherein the sensing arm is configured to guide sensing light, the reference arm is configured to guide reference light, and the waveguide interferometer is configured to interfere the sensing light with the reference light, and
wherein the waveguide interferometer and the microfluidic channel are configured to allow the sensing light to interact with the fluid and the micro-organisms in the sensing region of the microfluidic channel.

2. The sensor device according to claim 1, wherein the sensing arm comprises an optical waveguide such as a single-mode optical waveguide, the reference arm comprises an optical waveguide such as a single-mode optical waveguide, and the sensing and reference light each comprises a guided optical mode, such as a guided transverse magnetic (TM) optical mode, and, optionally, wherein the waveguide interferometer and the microfluidic channel are configured to allow an evanescent field of the guided optical mode to interact with the micro-organisms in the sensing region.

3. The sensor device according to claim 1, wherein the waveguide interferometer and the microfluidic channel are configured to allow the reference light to interact with the fluid and the micro-organisms in the microfluidic channel and/or wherein the waveguide interferometer and the microfluidic channel are configured for exposure of the reference arm of the waveguide interferometer to the fluid and the micro-organisms.

4. The sensor device according to claim 1, wherein the waveguide interferometer and the microfluidic channel are configured so as to prevent the reference light from interacting with the fluid and the micro-organisms in the microfluidic channel and/or wherein the waveguide interferometer and the microfluidic channel are configured so as to prevent exposure of the reference arm to the fluid and the micro-organisms.

5. The sensor device according to claim 4, comprising a cover layer or mask located between the reference arm and the microfluidic channel, which cover layer or mask prevents the reference light from interacting with the fluid and the micro-organisms in the microfluidic channel and/or prevents exposure of the reference arm to the fluid and the micro-organisms.

6. The sensor device according to any preceding claim 1, comprising:
a plurality of waveguide interferometers, each waveguide interferometer having a sensing arm and a reference arm;
a plurality of microfluidic channels for the fluid and the micro-organisms, and
a trapping arrangement in each microfluidic channel for physically trapping the micro-organisms when the fluid flows along the corresponding microfluidic channel so as to concentrate the micro-organisms in a corresponding sensing region,
wherein each sensing arm is configured to guide sensing light, each reference arm is configured to guide reference light, and each waveguide interferometer is configured to interfere the corresponding sensing light with the corresponding reference light, and
wherein the waveguide interferometers and the microfluidic channels are configured to allow the sensing light in the sensing arm of each waveguide interferometer to interact with the fluid and the micro-organisms in the sensing region of the corresponding microfluidic channel.

7. The sensor device according to claim 6, wherein one of the microfluidic channels contains a first micro-organism growth-inhibiting substance, and, optionally, wherein:
one or more of the other microfluidic channels contains a corresponding micro-organism growth-inhibiting substance which is different to the first micro-organism growth-inhibiting substance, and/or
one or more of the other microfluidic channels does not contain any micro-organism growth-inhibiting substance.

8. The sensor device according to any preceding claim 1, wherein each microfluidic channel comprises a well for receiving a micro-organism growth-inhibiting substance at a position located upstream from the corresponding sensing region in the same microfluidic channel.

9. The sensor device according to claim 1, wherein each trapping arrangement is located downstream from the sensing arm of the corresponding waveguide interferometer or wherein each trapping arrangement is located at the same position along the corresponding microfluidic channel as the sensing arm of the corresponding waveguide interferometer, for example wherein each trapping arrangement is located adjacent to the sensing arm of the corresponding waveguide interferometer.

10. The sensor device according to claim 1, wherein the trapping arrangement in each microfluidic channel defines one or more gaps which are configured to allow fluid flow to pass the trapping arrangement but to prevent micro-organisms from passing the trapping arrangement, for example wherein each waveguide interferometer is defined on, or adjacent, a surface of a photonic chip defining the one or more waveguide interferometers, and the trapping arrangement defines one or more gaps between the trapping arrangement and the surface of the photonic chip, wherein each gap is configured to allow fluid flow to pass through the gap between the trapping arrangement and the surface of the photonic chip but to prevent micro-organisms from passing through the gap between the trapping arrangement and the surface of the photonic chip.

11. The sensor device according to claim 1, wherein the trapping arrangement in each microfluidic channel comprises a plurality of trapping features, wherein the trapping features are configured to physically trap the micro-organisms when the fluid flows along the microfluidic channel,

12. The sensor device according to claim 1, wherein the trapping arrangement in each microfluidic channel comprises one or more rows of trapping features and, optionally, wherein the trapping arrangement in each microfluidic channel comprises two or more staggered rows of trapping features.

13. The sensor device according to claim 11, wherein the trapping features define one or more gaps which are configured to allow fluid flow to pass the trapping features but to prevent micro-organisms from passing the trapping features, for example wherein each waveguide interferometer is defined on, or adjacent, a surface of a photonic chip defining the one or more waveguide interferometers and each trapping feature defines one or more gaps between the trapping feature and the surface of the photonic chip, wherein each gap is configured to allow fluid flow to pass through the gap between the trapping feature and the surface of the photonic chip but to prevent micro-organisms from passing through the gap between the trapping feature and the surface of the photonic chip.

14. The sensor device according to claim 11, wherein each trapping feature comprises a trap configured to physically trap the micro-organisms when the fluid flows along the microfluidic channel, wherein each trap comprises one or more features extending into the corresponding microfluidic channel so as to define a bay in the corresponding microfluidic channel for accommodating one or more micro-organisms.

15. The sensor device according to claim 1, wherein the sensing arm of each waveguide interferometer is folded so that the sensing arm passes the corresponding sensing region of the corresponding microfluidic channel a plurality of times and/or wherein the reference arm of each waveguide interferometer is folded.

16. The sensor device according to claim 1, comprising a filtering arrangement in each microfluidic channel at a position located upstream from the corresponding sensing region, wherein the filtering arrangement is configured to trap debris or particulates which are greater in size than the micro-organisms, for example debris or particulates having a minimum dimension which is greater than a maximum dimension of the micro-organisms and, optionally, wherein each filtering arrangement comprises one or more projections extending into the corresponding microfluidic channel, wherein the one or more projections define at least one gap which exceeds a maximum dimension of the micro-organisms.

17. A sensor device for use in sensing a change in a concentration of micro-organisms, the sensor device comprising:
a plurality of waveguide interferometers, each waveguide interferometer having a sensing arm and a reference arm; and
a plurality of microfluidic channels, each channel configured to accommodate a fluid containing micro-organisms,
wherein each sensing arm is configured to guide sensing light, each reference arm is configured to guide reference light, and each waveguide interferometer is configured to interfere the corresponding sensing light with the corresponding reference light, and
wherein each waveguide interferometer and the corresponding microfluidic channel are configured so that the sensing light of each waveguide interferometer interacts with a greater concentration of the micro-organisms-in the corresponding microfluidic channel than the corresponding reference light,
wherein one of the microfluidic channels contains a first micro-organism growth-inhibiting substance, and
wherein one or more of the other microfluidic channels contains a corresponding micro-organism growth-inhibiting substance which is different to the first micro-organism growth-inhibiting substance and/or one or more of the other microfluidic channels does not contain any micro-organism growth-inhibiting substance.

18. The reader apparatus for reading a sensor device according to claim 1, the reader apparatus comprising:
an optical source for emitting light to be coupled into each waveguide interferometer;
one or more optical detectors for detecting light output from each waveguide interferometer and generating a corresponding electrical signal; and
a controller for determining a change, or a rate of change, in the concentration of the micro-organisms in the sensing region of each microfluidic channel based on the evolution of the corresponding electrical signal over time.

19. The reader apparatus according to claim 18, wherein the controller is configured to determine a change, or a rate of change, in the concentration of the micro-organisms in the sensing region of the corresponding microfluidic channel from oscillations in the corresponding electrical signal.

20. The reader apparatus according to claim 19, wherein the controller is configured to determine the change, or a rate of change, in the concentration of the micro-organisms in the sensing region of the corresponding microfluidic channel from the frequency of the oscillations in the corresponding electrical signal.

21. The reader apparatus according to claim 19, wherein the controller is configured to determine a change, or a rate of change, in the concentration of the micro-organisms in the sensing region of one microfluidic channel containing a first micro-organism growth-inhibiting substance relative to a change, or a rate of change, in the concentration of the micro-organisms in the sensing region of a microfluidic channel containing a different micro-organism growth-inhibiting substance based on the oscillations in the electrical signal corresponding to the microfluidic channel containing the first micro-organism growth-inhibiting substance and the oscillations in the electrical signal corresponding to the microfluidic channel containing the different micro-organism growth-inhibiting substance.

22. The reader apparatus according to claim 19, wherein the controller is configured to determine a change, or a rate of change, in the concentration of the micro-organisms in the sensing region of each microfluidic channel containing a micro-organism growth-inhibiting substance relative to a change, or a rate of change, in the concentration of the micro-organisms in the sensing region of the microfluidic channel which does not contain any micro-organism growth-inhibiting substance based on the oscillations in the electrical signal corresponding to each microfluidic channel containing a micro-organism growth-inhibiting substance and the oscillations in the electrical signal corresponding to the microfluidic channel which does not contain any micro-organism growth-inhibiting substance.

23. A sensing method for sensing a change in a concentration of micro-organisms, the sensing method comprising:
passing a fluid containing micro-organisms along a microfluidic channel;
physically trapping micro-organisms when the fluid flows along the microfluidic channel so as to concentrate the micro-organisms in a sensing region of the microfluidic channel;
propagating sensing light along a sensing arm of a waveguide interferometer;
propagating reference light along a reference arm of the waveguide interferometer; and
interfering the sensing light with the reference light,
wherein the waveguide interferometer and the microfluidic channel are configured so that the sensing light interacts with the fluid and the micro-organisms in the sensing region of the microfluidic channel.

24. A sensing method for sensing a change in a concentration of micro-organisms, the sensing method comprising:
passing a fluid containing micro-organisms along a plurality of microfluidic channels;
propagating sensing light along a sensing arm of each waveguide interferometer of a plurality of waveguide interferometers;
propagating reference light along a reference arm of each waveguide interferometer of the plurality of waveguide interferometers;
interfering the sensing light with the corresponding reference light,
wherein each waveguide interferometer and the corresponding microfluidic channel are configured so that the sensing light of each waveguide interferometer interacts with a greater concentration of the micro-organisms in the corresponding microfluidic channel than the corresponding reference light,
wherein one of the microfluidic channels contains a first micro-organism growth-inhibiting substance, and
wherein one or more of the other microfluidic channels contains a corresponding micro-organism growth-inhibiting substance which is different to the first micro-organism growth-inhibiting substance and/or one or more of the other microfluidic channels does not contain any micro-organism growth-inhibiting substance.

25. The sensor device according to claim 1, wherein at least one of:
the fluid comprises a bodily fluid such as urine, blood, saliva, or sputum;
the micro-organisms comprise at least one of bacteria, fungi or algae; or
the micro-organisms comprise bacteria and each micro-organism growth-inhibiting substance comprises an antibiotic.

26. The reader apparatus according to claim 18, wherein at least one of:
the fluid comprises a bodily fluid such as urine, blood, saliva, or sputum;
the micro-organisms comprise at least one of bacteria, fungi or algae; or
the micro-organisms comprise bacteria and each micro-organism growth-inhibiting substance comprises an antibiotic.

27. The sensing method according to claim 23, wherein at least one of:
the fluid comprises a bodily fluid such as urine, blood, saliva, or sputum;
the micro-organisms comprise at least one of bacteria, fungi or algae; or
the micro-organisms comprise bacteria and each micro-organism growth-inhibiting substance comprises an antibiotic.
